(19) United States
(12) Patent Application Publication
(10) Pub. No.: US 2024/0342400 A1
(43) Pub. Date: Oct. 17, 2024

(54) THERAPEUTIC DE-ADDICTION SYSTEM AND METHODS

(71) Applicant: Flik Therapeutics, Inc., Philadelphia, PA (US)

(72) Inventor: Brendan Heiden, Philadelphia, PA (US)

(73) Assignee: Flik Therapeutics, Inc., Philadelphia, PA (US)

(21) Appl. No.: 18/626,527

(22) Filed: Apr. 4, 2024

Related U.S. Application Data
(60) Provisional application No. 63/457,322, filed on Apr. 5, 2023.

(51) Int. Cl. A61M 11/04; A61K 31/137; A61K 31/465; A61K 31/498; A61M 15/06; G16H 20/10

(52) U.S. Cl. CPC A61M 11/042; A61K 31/137; A61K 31/465; A61K 31/498; A61M 11/047; A61M 15/06; G16H 20/10

(57) ABSTRACT

A smoking cessation system may include a nicotine delivery device to deliver a mixture in an aerosol form to a user, through pulmonary delivery. The mixture may include nicotine. The system may be prescription, over-the-counter, or non-prescription based. The smoking cessation system may also include a health hub to communicate with the nicotine delivery device and to control operation of the nicotine delivery device based on information. Such information may include, for example, information relating to the use of the nicotine delivery device by the user. One or more additional devices (e.g., a continuous monitoring device that detects nicotine content and use, including through metabolites such as cotinine or carbon monoxide) may communicate one or more items of the information to the health hub for the purpose of assisting the user to quit smoking.

### RELATED APPLICATIONS

This application claims priority (under 35 USC 119 (e)) to U.S. Provisional Application No. 63/457,322, filed Apr. 5, 2023, the entire contents of which are incorporated herein by reference in their entirety.

### BACKGROUND

This invention relates to a therapeutic and, more particularly, to a therapeutic de-addiction system and methods in support of such systems.

Therapeutics for de-addiction can address individual and societal issues. For example, smoking presents an enormous cost to individual health, as well as to the global economy. Smoking-related illness takes a toll on countless families each year, and the healthcare costs are a substantial burden to public health. In addition, cigarettes and their filters have a negative impact on environmental pollution. Most smokers experience pharmacological addiction (an addiction to nicotine), psychosocial addiction (a susceptibility to social cues to smoke), and sensory addiction (an affinity to the sensory experience of smoking). While a majority of individuals who smoke indicate a desire to quit, the current suite of tobacco treatment therapies have proven inadequate, with low efficacy. Therefore, most individuals make several quit attempts before ultimately dropping the habit. Prior treatments to help with smoking cessation have included nicotine replacement therapy and other medications which are often paired with behavioral support interventions.

### SUMMARY

According to one or more embodiments, a therapeutic de-addiction system includes a smoking cessation system that may include a nicotine delivery device to deliver a mixture in an aerosol form to a user, through pulmonary delivery. The mixture may include nicotine. The system may be prescription, over-the-counter, or non-prescription based. The smoking cessation system may also include a health hub to communicate with the nicotine delivery device which may control the operation of the nicotine delivery device based on information, or vice versa. Such information may include, for example, information relating to the use of the nicotine delivery device by the user. One or more additional devices (e.g., a continuous monitoring device that detects nicotine content and use, including through metabolites such as cotinine or carbon monoxide) may communicate one or more items of the information to the health hub for the purpose of assisting the user to quit smoking.

According to another embodiment, a nicotine delivery device includes an aerosolization system using one or more methods (e.g., heating, soft-mist, nebulizer, or piezoelectric methods) to aerosolize a mixture for consumption by a user. A communication interface communicates with a health hub that receives information about the user. A processor inhibits the consumption by the user based on a control signal from the health hub.

According to yet another embodiment, a method implemented by a nicotine delivery device includes identifying a pod (e.g., a vial containing a pharmaceutical solution) that is interfaced with the nicotine delivery device, the pod storing a mixture to be aerosolized by the nicotine delivery device. The method also includes determining, based on an identity of the pod, a dosage of the mixture stored by the pod and determining whether the dosage is permitted for a user of the nicotine delivery device. Use of the nicotine delivery device is inhibited based on determining that the dosage is higher than a permitted dosage for the user.

According to one or more embodiments, a smoking cessation system includes a nicotine delivery device to deliver a mixture in an aerosol form to a user, the mixture including nicotine, varenicline, or bupropion, and a health hub to communicate with the nicotine delivery device and to control operation of the nicotine delivery device based on information. One or more additional devices communicate one or more items of the information to the health hub.

Additionally, according to one or more embodiments, the one or more additional devices includes a smartphone used by the user.

Additionally, according to one or more embodiments, the one or more items includes a location of the user.

Additionally, according to one or more embodiments, the health hub provides one or more messages to the user via the smartphone based on the location.

Additionally, according to one or more embodiments, the one or more items includes biometric data from the user, wherein the health hub controls the nicotine delivery device to suspend operation based on determining that the user is not an authorized user according to the biometric data.

Additionally, according to one or more embodiments, the biometric data is a thumbprint or facial recognition.

Additionally, according to one or more embodiments, the health hub is an application on the smartphone.

Additionally, according to one or more embodiments, the one or more additional devices includes a monitoring device.

Additionally, according to one or more embodiments, the monitoring device measures a concentration of cotinine of the user or carbon monoxide exhaled by the user and provides the concentration of cotinine or the carbon monoxide and a date and time of measurement to the health hub.

Additionally, according to one or more embodiments, the health hub controls the nicotine delivery device to suspend operation based on determining that the concentration of cotinine or the carbon monoxide is higher than an allowed concentration.

Additionally, according to one or more embodiments, the one or more additional devices includes a testing device configured to obtain testing information from the user as the one or more items of the information.

Additionally, according to one or more embodiments, the health hub controls the nicotine delivery device based on the testing information.

Additionally, according to one or more embodiments, the testing device obtains the testing information from plasma, blood, urine, saliva, interstitial fluid, skin, sweat, breath, hair, nails, tears, ear canal, nasal passage, oral passage, anal secretion or excrement, or stool of the user, and the testing information indicates a level of nicotine, cotinine, tans-3-hydroxycotinine, nornicotine, or carbon monoxide.

Additionally, according to one or more embodiments, the smoking cessation system includes a pod configured to fit within the nicotine delivery device. The mixture is stored in the pod.

Additionally, according to one or more embodiments, the pod includes an identifier indicating an identity of the pod, the identity indicating a dosage of the mixture in the pod.

Additionally, according to one or more embodiments, the nicotine delivery device obtains the identity via the identifier and to suspend operation based on the dosage of the mixture in the pod, as indicated by the identifier, being higher than an allowed dosage.

Additionally, according to one or more embodiments, the smoking cessation system includes a healthcare provider interface to communicate with the health hub, wherein the allowed dosage is based on input from the healthcare provider interface.

Additionally, according to one or more embodiments, the nicotine delivery device obtains an identity of the user and suspends operation based on the identity indicating the user as other than an authorized user.

Additionally, according to one or more embodiments, the nicotine delivery device provides additional information to the health hub.

Additionally, according to one or more embodiments, the nicotine delivery device monitors a number of actuations implemented by the user and provides the number of actuations as the additional information.

Additionally, according to one or more embodiments, the nicotine delivery device determines a first amount of the mixture consumed by the user and a second amount of the mixture remaining and provides the first amount or the second amount as the additional information.

According to one or more embodiments, a nicotine delivery device includes an aerosolization system to aerosolize a mixture for consumption by a user, and a communication interface to communicate with a health hub that receives information about the user. A processor inhibits the consumption by the user based on a control signal from the health hub.

Additionally, according to one or more embodiments, the aerosolization system includes a mesh nebulizer, pressurized metered-dose inhaler, or soft-mist inhaler.

Additionally, according to one or more embodiments, the nicotine delivery device also includes one or more measurement components.

Additionally, according to one or more embodiments, one of the one or more measurement components includes a temperature sensor.

Additionally, according to one or more embodiments, the aerosolization system includes a temperature control system, and the temperature sensor triggers the temperature control system based on detecting an overheat condition.

Additionally, according to one or more embodiments, one or more of the one or more measurement components monitors an amount of the mixture consumed by the user and a number of actuations of the nicotine delivery device corresponding to dosage of aerosolized mixture taken by the user.

Additionally, according to one or more embodiments, the nicotine delivery device also includes a pod interface and a pod identity reader, wherein the nicotine delivery device interfaces with a pod storing the mixture at the pod interface and the pod identity reader determines an identity of the pod.

Additionally, according to one or more embodiments, the identity of the pod is associated with a dosage of the mixture stored by the pod and the communication interface is used to communicate the identity of the pod to the health hub.

Additionally, according to one or more embodiments, the pod identity reader is a barcode reader to read a barcode on the pod.

Additionally, according to one or more embodiments, the nicotine delivery device also includes a biometric reader to obtain biometric information from the user.

Additionally, according to one or more embodiments, the biometric reader is a thumbprint reader.

Additionally, according to one or more embodiments, the nicotine delivery device inhibits operation based on an identity of the user determined with the biometric information.

According to one or more embodiments, a method implemented by a nicotine delivery device includes identifying a pod that is interfaced with the nicotine delivery device, the pod storing a mixture configured to be aerosolized by the nicotine delivery device, and determining, using a processor and based on an identity of the pod, a dosage of the mixture stored by the pod. The method also includes determining whether the dosage is permitted for a user of the nicotine delivery device, and inhibiting use of the nicotine delivery device based on determining that the dosage is higher than a permitted dosage for the user.

Additionally, according to one or more embodiments, the identifying the pod includes using a barcode reader to read a barcode on the pod or a radio frequency identifier (RFID) reader to read an RFID on the pod.

Additionally, according to one or more embodiments, the method also includes obtaining an identity of the user and determining if the user is an authorized user.

Additionally, according to one or more embodiments, the obtaining the identity of the user includes using a thumbprint reader to read a thumbprint of the user.

Additionally, according to one or more embodiments, the method also includes inhibiting use of the nicotine delivery device based on determining that the user is not the authorized user.

Additionally, according to one or more embodiments, the method also includes monitoring information during use based on determining that the dosage is the permitted dosage for the user.

Additionally, according to one or more embodiments, the monitoring the information includes monitoring usage information.

Additionally, according to one or more embodiments, the method also includes communicating the usage information to a health hub that monitors a progress of the user in a smoking cessation program.

Additionally, according to one or more embodiments, the monitoring the information includes monitoring temperature within the nicotine delivery device.

According to one or more embodiments, a method implemented by a health hub includes obtaining information pertaining to a nicotine delivery device or a user of the nicotine delivery device, and determining, using a processor, whether the information indicates one or more issues. The one or more issues include an unauthorized use of the nicotine delivery device. The method also includes controlling an operation of the nicotine delivery device via a control signal based on determining the unauthorized use of the nicotine delivery device.

Additionally, according to one or more embodiments, the obtaining the information includes obtaining an indication that the user is not an authorized user of the nicotine delivery device.

Additionally, according to one or more embodiments, the obtaining the information includes obtaining an indication that a dosage of a mixture to be aerosolized by the nicotine delivery device is higher than an authorized dosage for the user.

Additionally, according to one or more embodiments, the obtaining the information includes obtaining an indication that testing information obtained from the user makes use of the nicotine delivery device unauthorized.

Additionally, according to one or more embodiments, the method also includes determining that the user should be awarded a reward for adherence to prescribed use of the nicotine delivery device based on the information.

Additionally, according to one or more embodiments, the method also includes determining that the user should be provided with encouraging messages to continue with a cigarette cessation program based on the information.

Additionally, according to one or more embodiments, the method also includes permitting use of the nicotine delivery device and updating a healthcare provider with the information.

The foregoing has outlined some of the pertinent features of the disclosed subject matter. These features are merely illustrative.

### BRIEF DESCRIPTION OF THE DRAWINGS

The examples described throughout the present document will be better understood with reference to the following drawings and descriptions. In the figures, like-referenced numerals designate corresponding parts throughout the different views.

FIG. 1 is a block diagram of a smoking cessation system according to an exemplary embodiment;

FIG. 2 shows aspects of an exemplary carrying case for the nicotine delivery device;

FIG. 3 shows an interface between the nicotine delivery device and an exemplary carrying case according to exemplary embodiments;

FIG. 4 shows an interface between the nicotine delivery device and a pod according to exemplary embodiments;

FIG. 5 shows the interface between the nicotine delivery device and the pod from a different perspective that the one shown in FIG. 4;

FIG. 6 shows an exemplary package of pods;

FIG. 7 is a block diagram of the nicotine delivery device according to exemplary embodiments;

FIG. 8 is a process flow of a method implemented by the nicotine delivery device according to exemplary embodiments;

FIG. 9 is an exemplary illustration of a continuous monitoring device;

FIG. 10 is a block diagram of the health hub according to exemplary embodiments; and

FIG. 11 is a process flow of a method implemented by the health hub according to exemplary embodiments.

### DETAILED DESCRIPTION

Reference will now be made to the drawings to describe the present disclosure in detail. It will be understood that the drawings and exemplified embodiments are not limited to the details thereof. Modifications may be made without departing from the spirit and scope of the disclosed subject matter.

As noted, smoking has negative effects on the individual smoker, on society as a whole, and on the environment. While most smokers indicate a desire to quit, many find prior approaches to treatment to be unpleasant and ineffective. It is appreciated that these prior approaches do not dynamically adjust treatment or provide other interventions in real-time. These prior approaches also fail to address the multidimensional aspects of addiction, including pharmacologic, psychosocial, and habitual addiction.

Embodiments detailed herein relate to a smoking cessation system. Aspects of the smoking cessation system provide real-time monitoring of progress and engagement with the user. An exemplary aspect of the smoking cessation system involves an inhaled nicotine delivery device referred to as an inhaled nicotine replacement therapy (iNRT). The device may facilitate control of the dosage of materials provided to the user. The device may also facilitate verification of the user (to ensure that only the intended user is accessing the iNRT) and/or consideration of user location (to ensure that the user is not using the iNRT in a school or hospital, for example).

Another exemplary aspect of the smoking cessation system involves monitoring information about the user. The information may include measurements of material intake so that subsequent dosage may be adjusted. The information may also include location or behavioral information so that an AI-based algorithm (e.g., machine learning application) may intervene to prevent potential cigarette and/or vaping device and/or recreational tobacco product use. In exemplary embodiments, statistical models may be used to determine whether the user is on a path to successfully quit smoking. Based on the various aspects, pharmacological, psychosocial, and sensory addiction are addressed by the system described herein, its components and associated software, to increase a likelihood of success of the smoking cessation system.

The smoking cessation system may be prescribed or suggested by a physician or other healthcare provider (e.g., physician's assistant, nurse practitioner, registered nurse) who may specify a starting dosage, for example. The starting dosage may be based on clinical testing of the user of the smoking cessation system, a clinical addiction survey, on behavioral information about the user (e.g., number of years as a smoker, number of packs smoked per day, a smoking addiction severity survey), and/or a “wash-in” period that monitors the amount of nicotine ingested through the iNRT. The smoking cessation system, when used as intended, decreases the dosage from that starting dosage in a gradual fashion via a series of pods that hold the material that will be aerosolized for inhalation by the user. By monitoring the user (e.g., biochemical verification, location, pod usage), dynamic adjustments may be made to the standard treatment program to accelerate or slow the program to ensure success. In addition, real-time interventions may be deployed to promote more productive behaviors. For example, encouraging messages may be provided to the user when they enter an environment, such as a gas station, in which recreational tobacco products are sold. Rewards, including behavioral and financial, may be given to the user when they adhere to the program. Additional behavioral support, including cognitive behavioral therapy techniques and other forms of behavioral therapy, may be initiated based on these communications.

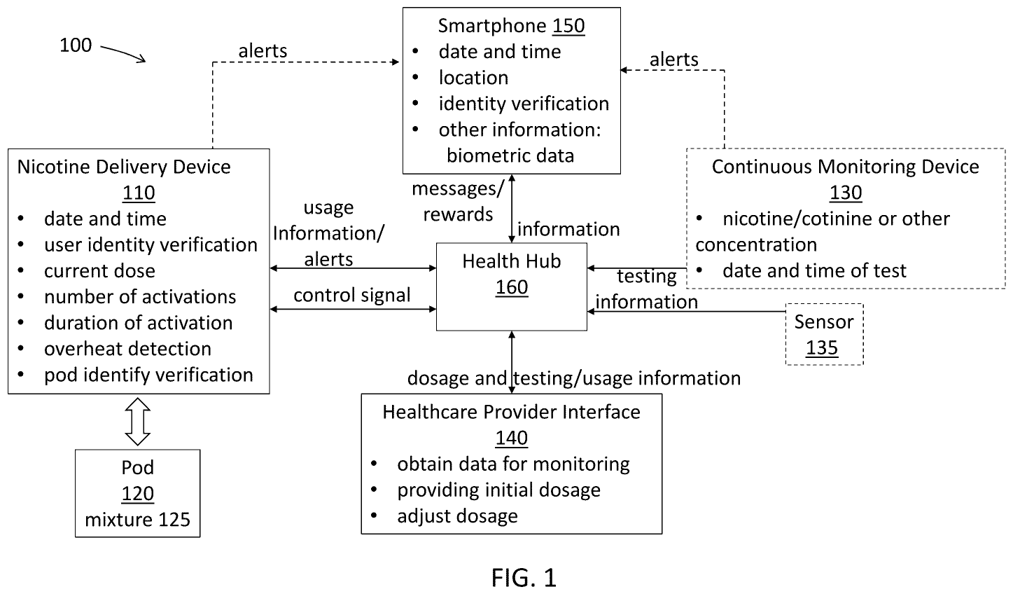

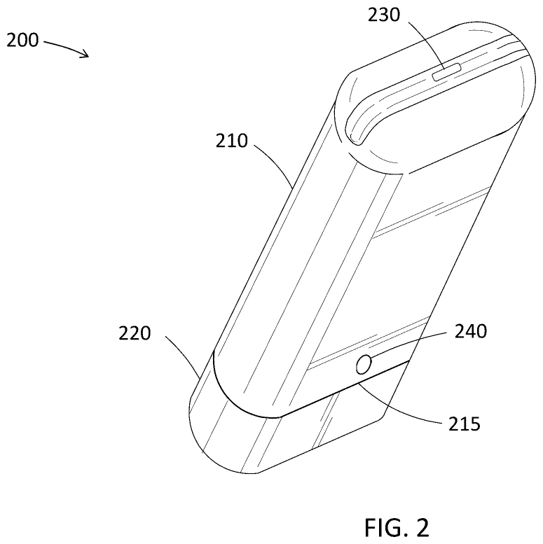

FIG. 1 is a block diagram of a smoking cessation system 100 according to one or more embodiments. As shown, the smoking cessation system 100 generally includes a nicotine delivery device 110, a pod 120, a continuous monitoring device 130 and/or a sensor 135 that differs from the continuous monitoring device 130, a healthcare provider interface 140, a smartphone 150, and a health hub 160, which may be an application implemented on the smartphone 150 according to exemplary embodiments. Each of the components of the smoking cessation system 100 is briefly described with reference to FIG. 1 and further detailed with reference to FIGS. 2-12.

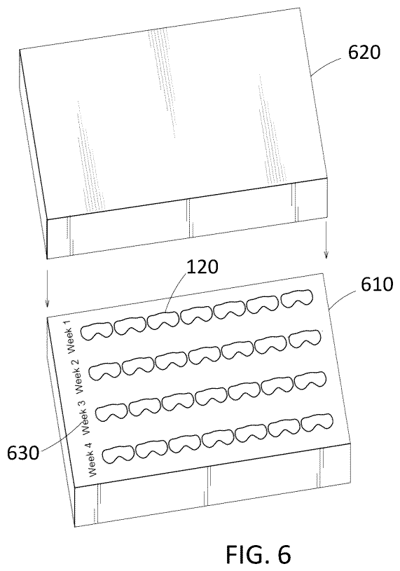

The nicotine delivery device 110 (e.g., an iNRT) of the smoking cessation system 100 interfaces with the pod 120 that holds a specific dose of a mixture 125. The pod 120, according to some embodiments, is not limited to a particular size or volume and is generally a vial or container that holds a pharmaceutical formulation or, more generally, a mixture 125 that is aerosolized by the iNRT. The mixture 125 may include the following materials: water, nicotine (e.g., 0 to 60 milligrams per milliliter (mg/mL)), an organic acid salt buffer (such as, for example, benzoic, lactic, levulinic, tartaric, pyruvic, or citric acid), propylene glycol, glycerol, other excipients, and flavoring (e.g., menthol, mint). The mixture may also include other active pharmaceutical ingredients for the purposes of smoking cessation such as varenicline or bupropion. The exemplary materials and the exemplary amounts are not intended to limit either the constituent components of the mixture 125 or their quantities, and it is appreciated that other mixture types and quantities may be used. As further discussed with reference to FIG. 6, different pods 120 may hold mixtures with different dosages of the constituent components. The nicotine delivery device 110 and pod 120 are further discussed with reference to FIGS. 2-8.

As indicated in FIG. 1, the nicotine delivery device 110 may provide information (e.g., usage information indicating usage of the nicotine delivery device 110 by the user, alerts regarding a state of the nicotine delivery device 110) to and obtain control signals from the health hub 160. In some embodiments, the nicotine delivery device 110 may control aspects of the health hub 160 and/or operate independently in addition to or instead of via control signals from the health hub 160. In addition, the nicotine delivery device 110 may optionally provide alerts to the smartphone 150 to immediately alert the user (e.g., of an overheat condition, system error).

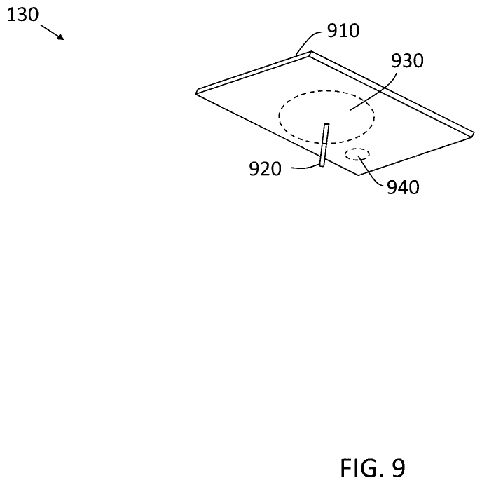

In exemplary embodiments, the smoking cessation system 100 may include a continuous monitoring device 130 which is used to determine whether the user is using nicotine (in the form of the nicotine delivery device 110 or otherwise) and the level of use. The monitoring may be periodic or at predefined times, for example. The continuous monitoring device 130 (e.g., a continuous nicotine monitor (CNM)) may be a wearable monitor, as discussed with reference to FIG. 9. The continuous monitoring device 130 may additionally or alternately include a carbon monoxide sensor.

As shown in FIG. 1, additionally or alternately, one or more other sensors 135 may be used for biochemical monitoring and/or verification. A sensor 135 may analyze plasma, blood, urine, saliva, interstitial fluid, skin, sweat, breath, hair, nails, tears, ear canal, nasal passage, oral passage, anal secretion or excrement, or stool of the user, and may provide a level of nicotine, cotinine, tans-3-hydroxycotinine, nornicotine, or other nicotine metabolite or smoking byproduct (e.g., carbon monoxide). The exemplary measurements are not intended to limit any additional or alternate testing procedure that may be implemented or testing information that may be obtained. For example, the sensor 135 may include an ex vivo handheld device which obtains the testing information from plasma, blood, urine, saliva, interstitial fluid, skin, sweat, breath, hair, nails, tears, car canal, nasal passage, oral passage, anal secretion or excrement, or stool of the user, and the testing information indicates a level of nicotine, cotinine, tans-3-hydroxycotinine, nornicotine, carbon monoxide, or some other analyte.

The user may be notified by the health hub 160 (e.g., via the smartphone 150) to use the sensor 135. This type of requested screening may happen randomly, for example, with results provided to the health hub 160. Data from these screenings may then be used to guide the treatment program further (e.g., behavioral support, financial rewards, etc.). Thus, an exemplary difference between a continuous monitoring device 130 and a sensor 135 may be that the continuous monitoring device 130 may passively obtain data (e.g., nicotine and/or carbon monoxide level) on a regular basis while a sensor 135 may require active use. In some embodiments, another sensor 135 may be a handheld or non-invasive wearable device that analyzes a biomarker of smoking cessation success. For example, a user may be prompted by the health hub 160 to use this other sensor 135, which may be a handheld device into which the user exhales, and the presence or absence of various substances in the breath may be calculated (e.g., carbon monoxide).

As indicated in FIG. 1, the continuous monitoring device 130 and/or other sensor 135 may provide testing information to the health hub 160. This information may be used by the health hub 160 to control subsequent use of the nicotine delivery device 110. For example, if testing information from the continuous monitoring device 130 and/or other sensor 135 indicates that a user's cotinine and/or carbon monoxide levels are higher than they should be if the cessation program and dosage were being followed properly, the health hub 160 may disable the nicotine delivery device 110 and/or take another action. In addition, the user's healthcare provider may be notified via the healthcare provider interface 140. As indicated in FIG. 1, the continuous monitoring device 130 may optionally provide alerts to the user via the smartphone 150. An exemplary alert may indicate that a level of a monitored parameter was detected to be above a threshold value and the user should contact their healthcare provider.

The healthcare provider interface 140 provides a connection to the healthcare provider who may manage the cessation process for the user. This healthcare provider may have prescribed or suggested the nicotine delivery device 110 and set the dosage schedule, which determines the mixture 125 of the pods 120 provided to the user. According to different exemplary embodiments, the healthcare provider interface 140 may include a display of information for the healthcare provider regarding the user's progress in quitting smoking. Alerts, controls, and other functions may also be part of the healthcare provider interface 140. As indicated in FIG. 1, the healthcare provider interface 140 may communicate with the health hub 160 to provide dynamic dosing adjustments and obtain usage information and/or testing information, which may be provided to the health hub 160 by the continuous monitoring device 130 or other sensor 135. An alert issued by or to the healthcare provider interface 140 may indicate that the healthcare provider should contact the user, for example.

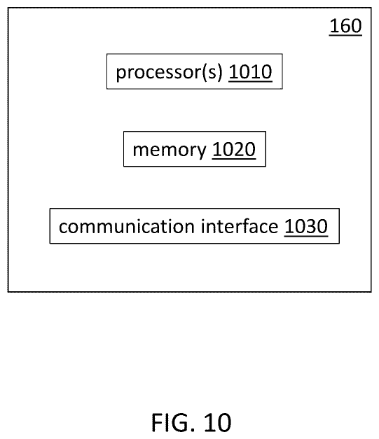

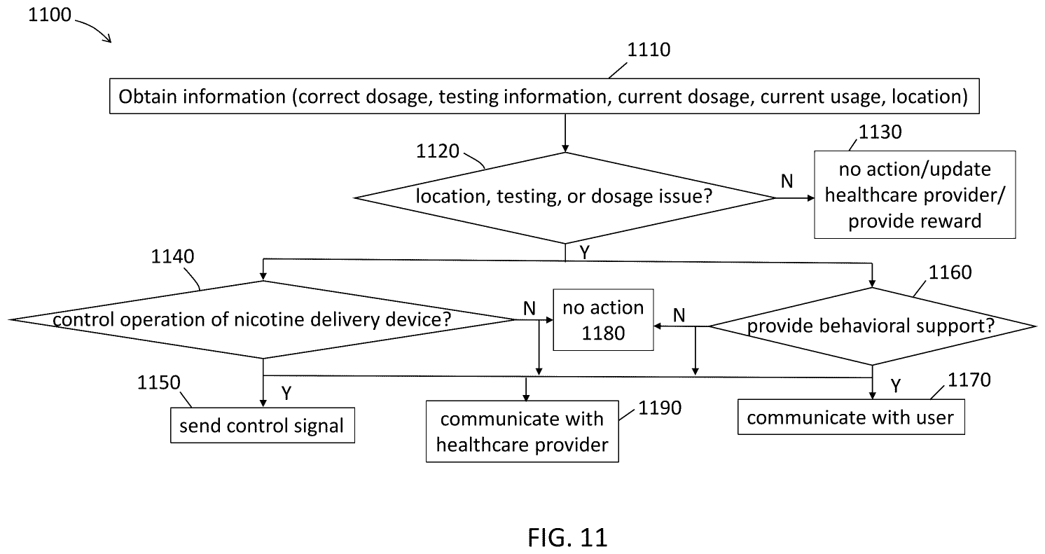

The smartphone 150 may be used to verify the identification of the user of the nicotine delivery device 110. For example, facial recognition may be used via the smartphone 150 and health hub 160 (e.g., photo acquired by camera of the smartphone 150 and facial feature analysis performed by aspects of the health hub 160) to permit/disallow use of the nicotine delivery device 110 based on determining whether the user is an authorized user. The smartphone 150 may also obtain the location of the user. As indicated in FIG. 1, the smartphone 150 may provide the identification and location information, as well as additional information, to the health hub 160. Examples of additional information include biometric information such as the user's pulse or wakefulness. Wakefulness may be determined based on other activity on the smartphone 150 or movement of the smartphone 150, for example. As also indicated, the health hub 160 may provide messages or rewards to the user via the smartphone 150. These messages or rewards may encourage the user to adhere to the prescribed cessation program and dosage or allow the user to see information provided to the health hub 160 by other components of the smoking cessation system 100. The health hub 160 may maintain various dashboards (e.g., medical, behavioral, psychological, social, financial). Patients or users can access these dashboards through the health hub 160, for example, to communicate with their healthcare provider or to participate in a social media type community. Additionally, a healthcare provider may access one or more of the dashboards via the healthcare provider interface 140. The health hub 160 is further detailed with reference to FIGS. 10 and 11.

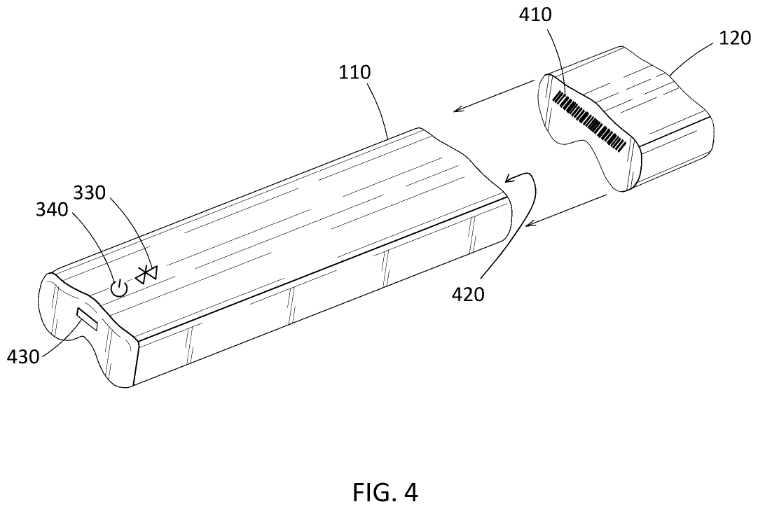

FIGS. 2-8 detail aspects of the nicotine delivery device 110 and pod 120 according to exemplary embodiments. FIG. 2 shows aspects of an exemplary carrying case 200 for the nicotine delivery device 110 and/or replacement pods 120 containing various mixture 125. The carrying case 200 includes a case body 210 and a case cover 220. The case cover 220 may be hinged such that it separates from the case body 210 at a hinged opening 215. The hinged opening 215 may replicate the opening of a pack of cigarettes, for example, to address sensory addiction. The position of the carrying case 200 shown in FIG. 2 exposes a charging port 230 (e.g., USB-C or other standard interface), which is associated with a charging indicator light 240, indicating a state of charging. This charging port 230 may line up with a charging port 430 (FIG. 4) of the nicotine delivery device 110 so that the nicotine delivery device 110 may be charged while it is stored in the carrying case 200. In some embodiments, the carrying case 200 may include a separate battery and charging components for the nicotine delivery device 110.

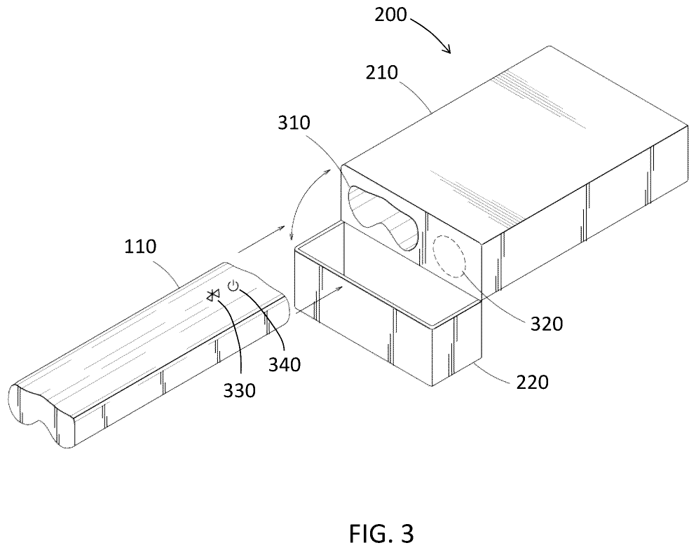

FIG. 3 shows an interface between the nicotine delivery device 110 and an exemplary carrying case 200 according to exemplary embodiments. While the exemplary carrying case 200 shown in FIG. 2 has rounded edges, the carrying case 200 shown in FIG. 3 is rectangular in shape. These two examples are not intended to limit other shapes for the carrying case 200. The case cover 220 is shown in an open position in FIG. 3, revealing a molded holder 310 for the nicotine delivery device 110 and a storage 320 for pods 120. The nicotine delivery device includes a Bluetooth communication indicator light 330, indicating whether communication via the Bluetooth standard is enabled, and a power indicator light 340, indicating whether the nicotine delivery device 110 is powered on.

FIG. 4 shows an interface between the nicotine delivery device 110 and a pod 120 according to exemplary embodiments. The pod 120 may be made from shatter-proof, high-grade glass and may be covered with plastic casing or the like to prevent photosensitivity reactions in the mixture 125. The pod 120 may include different compartments to keep components of the mixture 125 separated (e.g., nicotine-containing solution separate from other nicotine-free excipients). The components may be aerosolized using one or more methods (e.g., heating, soft-mist, nebulizer, or piezoelectric methods). The components of the mixture may be pre-mixed or stored in separate chambers and mixed only after the pod 120 is interfaced with the nicotine delivery device 110.

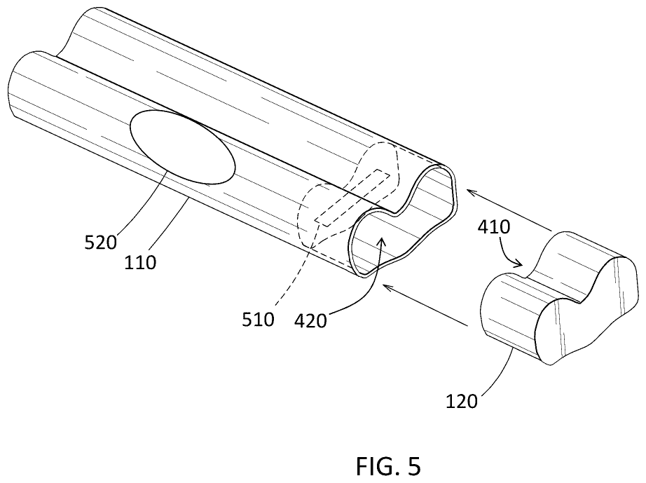

The pod 120 includes an identifier 410, which is a barcode in the exemplary embodiment shown in FIG. 4. The identifier 410 may be a radio frequency identifier (RFID) or another machine-readable identifier according to alternate embodiments. The nicotine delivery device 110 includes a pod interface 420 to attach to the pod 120. In the exemplary embodiment shown in FIGS. 4 and 5, the pod interface 420 is a cavity into which the pod 120 may be inserted entirely or partially. A groove, magnet, or other mechanism may secure the pod 120 in place.

In alternate embodiments, the pod interface 420 may be positioned on an outer surface of the nicotine delivery device 110 such that the pod 120 attaches to an outside of the nicotine delivery device 110. As indicated in FIG. 5, the nicotine delivery device 110 may include a pod identity reader 510 to read the identifier 410 on the pod 120. In the exemplary case of the pod 120 including a barcode, the pod identity reader 510 may be a barcode reader. The nicotine delivery device 110 is also shown to include the charging port 430 (e.g., USB-C), which may align with the charging port 230 of the carrying case 200, as shown in FIG. 2.

FIG. 5 shows the interface between the nicotine delivery device 110 and the pod 120 from a different perspective than the one shown in FIG. 4. In the view of FIG. 5, the cavity that represents the pod interface 420 of the nicotine delivery device 110 is visible. As indicated, a pod identity reader 510 (e.g., barcode reader, RFID reader) may be disposed in the cavity (pod interface 420) to read the identifier 410 of the pod 120. A pod 120 that does not have an identifier 410 that can be read by the pod identity reader 510 (e.g., a third-party pod) may not be allowed to interface with the nicotine delivery device 110. When the pod 120 does have an identifier 410 that can be read by the pod identity reader 510, the dosage, expiration date, and other information may be verified prior to allowing use of the mixture 125 in the pod 120.

The nicotine delivery device 110 is also shown to include a biometric reader 520, such as a thumbprint reader shown in FIG. 5. The biometric reader 520 may be used to verify that a person attempting to use the nicotine delivery device 110 is recognized as an authorized user. For example, the nicotine delivery device 110 may not be operable until the biometric reader 520 is used. While a biometric reader 520 is shown as an example, another approach, such as a combination lock, may be used to ensure that only an authorized user accesses the nicotine delivery device 110. As previously noted, other biometric data (e.g., facial recognition) may additionally or alternately used for verification of user authorization. Once authorized, the nicotine delivery device 110 may begin monitoring the use of the nicotine delivery device 110 (e.g., number of puffs, amount used) by the authorized user.

FIG. 6 shows an exemplary package of pods 120. A pod holder 610 may hold a set of pods 120. A pod holder cover 620 may fit onto the pod holder 610, as indicated. The pod labels 630 may indicate, to the user, which pods 120 should be used at a given time. For example, the pods 120 associated with the pod label 630 “Week 1” may have a higher dose of nicotine than the pods 120 associated with the pod label 630 “Week 4.” Based on the pod labels 630 shown in FIG. 6, for example, the user may understand that the cessation regimen will begin with the higher-dose pods 120 (“Week 1”) and gradually move to the lower-dose pods 120 (“Week 4”) on the designated schedule. The exemplary package is not intended to limit any additional or alternate dosing regimen (e.g., shorter or longer periods of time).

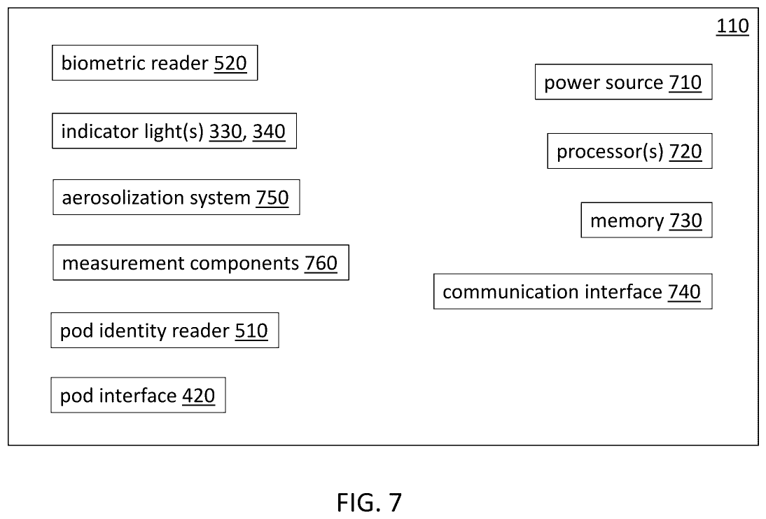

FIG. 7 is a block diagram of the nicotine delivery device 110 according to exemplary embodiments. The biometric reader 520 (e.g., thumbprint reader), indicator lights (e.g., Bluetooth communication indicator light 330, power indicator light 340), pod identity reader 510 (e.g., barcode reader, RFID reader), and pod interface 420 (e.g., cavity) of the nicotine delivery device 110 were previously discussed. In addition, the nicotine delivery device 110 may include a power source 710 (e.g., rechargeable battery), one or more processors 720, memory 730 (e.g., internal or removable), and a communication interface 740, such as the Bluetooth communication interface. The one or more processors may perform many of the functionalities that require other components. For example, biometric input obtained by the biometric reader 520 may be compared with biometric data stored in memory 730 by a processor 720 to determine if the biometric input is from a recognized or authorized user. As another example, biometric verification information (e.g., from a smartphone 150 based on facial recognition) may be received via the communication interface 740 and used by a processor 720 to initialize operation.

An aerosolization system 750 may include all the elements required to aerosolize the mixture 125. The aerosolization system 750 may include one or more known mechanisms of aerosolization, including a vibrating or ultrasonic mesh nebulizer, metered-dose inhaler (MDI) (e.g., pressurized MDI), a soft-mist inhaler technology (using colliding jets or Rayleigh jets), a heating element (with a temperature control system), or a capillary aerosol generator. One or more measurement components 760 may measure usage metrics and/or temperature. For example, the number of actuations implemented by the user may be counted. The number of actuations of the nicotine delivery device 110 refers to an exemplary way to determine dosage (i.e., amount) of the mixture 125 inhaled by the user. Additionally or alternately, an amount (e.g., volume) of the mixture 125 in the pod 120 during initial interface with the nicotine delivery device 110 may be compared with the amount prior to shutdown of the nicotine delivery device 110 to determine an amount used.

The processor 720 may monitor usage, status of the nicotine delivery device 110, and any additional information available to the nicotine delivery device 110 (e.g., device temperature, pod identity, error conditions) and convey that information or alerts generated from processing information via the communication interface 740. As indicated in FIG. 1, the nicotine delivery device 110 may communicate with the health hub 160 and may additionally communicate with the smartphone 150 or, when the health hub 160 is implemented on smartphone 150, with an application or different aspect implemented on the smartphone 150.

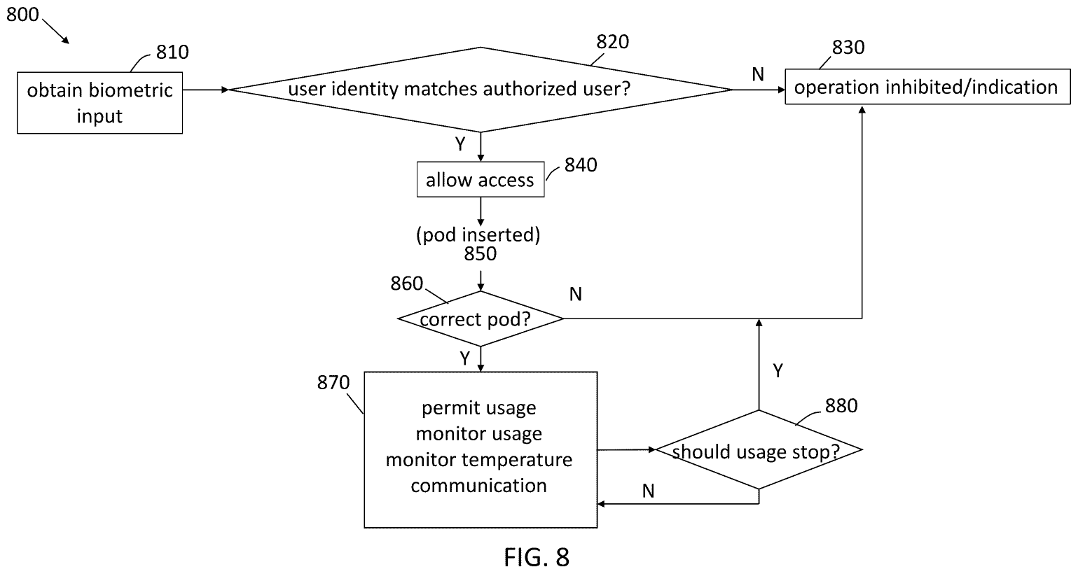

FIG. 8 is a process flow of a method 800 implemented by the nicotine delivery device 110 according to exemplary embodiments. At 810, obtaining biometric input is at the biometric reader 520. For example, a thumbprint may be obtained. As previously noted, biometric input may also refer to facial recognition mapped to identity or to authorization information communicated to the nicotine delivery device 110. At 820, a check is done of whether the user identity matches an authorized user. This check may involve the processor 720 comparing the biometric input obtained at 810 with biometric data stored in memory 730, for example. When authorization information is communicated to the nicotine delivery device 110 (e.g., from a smartphone 150/health hub 160), the check may involve the processor 720 obtaining that information. If the biometric input is determined not to be from an authorized user, then operation of the nicotine delivery device 110 may be inhibited and an indication may be provided. If the biometric input is determined to be from an authorized user, then allowing access, at 840, refers to allowing operation of the nicotine delivery device 110. This may mean that a pod 120 is allowed to be interfaced with the nicotine delivery device 110, at 850, for example.

At 860, a check is done of whether the pod 120 that was inserted (at 850) is a correct pod. The processor 720 may perform this check and may use the pod identity reader 510 and information stored in memory 730 from the health hub 160, for example. As an initial matter, the dosage of the inserted pod 120 may be determined by using the pod identity reader 510 to determine an identity of the pod 120. Information from the health hub 160 may map the identity of the pod 120 to its dosage. The health hub 160 may additionally provide information that indicates the correct dosage at the current stage of the cessation program. For example, the check (at 860) may be to determine whether the inserted pod 120 is a pod for week 2 of the cessation program because the current day and time corresponds with week 2 of the cessation program.

If the check (at 860) determines that the inserted pod 120 is not the correct pod 120, then further operation of the nicotine delivery device 110 may be inhibited and an indication may be provided (at 830). If the check (at 860) determines that the inserted pod 120 is an appropriate pod 120 for the current period of the cessation program, then, at 870, usage (e.g., aerosolization and inhalation of the mixture 125) of the nicotine delivery device 110 may be permitted. In addition, the nicotine delivery device 110 may perform monitoring of usage and temperature via measurement components 760 and communication with the health hub 160.

The communication may involve the nicotine delivery device 110 providing usage information to the health hub 110 and may additionally involve the health hub 160 communicating control signals to the nicotine delivery device 110. For example, based on the usage information from the nicotine delivery device 110 indicating excessive usage, according to a treatment plan provided to the health hub 160 via the healthcare provider interface 140, the health hub 160 may provide a control signal inhibiting further operation of the nicotine delivery device 110. When communication is received from the health hub 160, a check may be done, at 880, by the nicotine delivery device 110 to determine if usage should stop according to the communication. If the check (at 880) indicates that usage should stop according to the control signal from the health hub 160, then further operation of the nicotine delivery device 110 is inhibited and an indication is provided (at 830). If the check (at 880) indicates that usage may continue, then the processes at 870 are continued.

FIG. 9 is an exemplary illustration of a continuous monitoring device 130 (e.g., CNM). For example, the continuous monitoring device 130 may include a base 910 (e.g., an adhesive base) and a micropuncture needle 920 deployed into the subcutaneous tissue of the user. The interstitial fluid (ISF) may be analyzed by a sensor 930 to determine a concentration of nicotine, cotinine, or another marker in the user. Sensing by the continuous monitoring device 130 may be periodic and/or initiated by pressing a button 940 or by remote operation (e.g., via the smartphone 150), for example. The continuous monitoring device 130 may include a fluid reservoir, an interface with the body that includes an insertable fluid communication conduit (e.g., microdialysis catheter, microneedle), a pump to transport fluid between the ISF and the fluid reservoir, and a sensor for detecting the level of metabolites in the solution. The ISF may be extracted via either a microdialysis catheter, microneedle, or other means (e.g., reverse iontophoresis). That is, the continuous monitoring device 130 may use an ex vivo approach to extract ISF from peripheral tissues which is then transported to a metabolite sensor that is attached to the outside of the body (e.g., within the base 910). Exemplary metabolites include nicotine and cotinine. Additionally, the device may include a power source (e.g., battery), one or more processors, memory (e.g., internal or removable), and a communication interface, such as the Bluetooth communication interface. The one or more processors may perform many of the functionalities that require other components.

FIG. 10 is a block diagram of the health hub 160 according to exemplary embodiments. As previously noted, the health hub 160 may be an application implemented on the smartphone 150 according to an exemplary embodiment. In that case the components shown in FIG. 10 are components of the smartphone 150 that are used in the implementation of the functionality of the health hub 160. These components include one or more processors 1010, memory 1020, and a communication interface 1030. The memory 1020 may store instructions for implementation by the processor 1010 in addition to storing information provided by other components of the smoking cessation system 100. The communication interface 1030 may facilitate communication between the health hub 160 and the other components of the cigarette cessation system 100. The communication may be via a Bluetooth standard, for example.

The processor 1010 may implement artificial intelligence (AI) such as a machine learning algorithm, for example. The machine learning algorithm may use the information provided to the health hub 160. This information may include usage information from the nicotine delivery device 110, testing information from a continuous monitoring device 130 or other sensor 135, dosage information via the healthcare provide interface 140, location and user identity information from the smartphone 150 (or other applications implemented on the smartphone 150). As indicated in FIG. 1, all the information provided to the health hub 160 may include an associated date and time.

The machine learning algorithm may provide behavioral support to the user. This support may be in the form of notifications, voice alerts, graphs, badges, messages, or videos on the smartphone 150, for example. For example, if the information (e.g., use of the smartphone 150) indicates that the user is awake but has not used the nicotine delivery device 110 in more than a threshold period of time, a message discouraging cigarette use may be sent to the user to ensure that out-of-program use is not the cause. If information indicates that the user has stopped the program altogether, a message may be issued to the user to contact their healthcare provider. When the cessation program is being followed, according to the information, rewards may be issued in a gamification of the cessation effort. For example, if usage information provided by the nicotine delivery device 110 indicates that the user is using the device less frequently, incentives or rewards may be offered to the user. As another example, gamification may be employed as a distraction, based on the user's behavior or at predefined times, to help combat an urge to smoke. Additionally, the real-world data collected through the health hub 160 may be further quantified and stored in order to calculate subsequent environmental benefits of smoking cessation (e.g., carbon tax credits).

FIG. 11 is a process flow of a method 1100 implemented by the health hub 160 according to exemplary embodiments. At 1110, obtaining information refers to the different inputs to the health hub 160 such as, for example, usage information from the nicotine delivery device 110, testing information from a continuous monitoring device 130 or other sensor 135, dosage information via the healthcare provider interface 140, location, user identity, biometric and other information from the smartphone 150 (or other applications implemented on the smartphone 150).

At 1120, the processor 1010 of the health hub 160 determines if there is an issue based on any of the received information. Some exemplary issues have been discussed and are also included here. The information may be biometric information (e.g., from the smartphone 150) and usage information from the nicotine delivery device 110, which indicates that the user has been awake for more than a predefined length of time but has not interacted with the nicotine delivery device 110. In this case, the health hub 160 may detect or avert cigarette use by the user through messages, distraction, and the like. The information from the continuous monitoring device 130 or other sensor 135 may indicate lower concentrations of nicotine than expected at a given stage of the cessation program. The health hub 160 may process this information as indicative of faster than expected progress and provide rewards to the user (e.g., via the smartphone 150) in addition to informing the healthcare provider, via the healthcare provider interface 140, that a dosage review may be needed to further support faster cessation. These examples are not intended to limit other issues that may be identified by the health hub 160 based on the information received.

If the health hub 160 determines that there are no issues requiring additional action (e.g., the user is progressing on pace with the program), then, at 1130, the actions taken may include an update of the progress to the healthcare provider via the healthcare provider interface 140, and/or a reward to the user. If the health hub 160 determines that there are issues that may require additional actions, then checks are done at 1140 and at 1160.

At 1140, a check is done of whether the issue determined by the health hub 160 requires control of the operation of the nicotine delivery device 110. If so, a control signal is sent to the nicotine delivery device 110 at 1150. If not, no action is taken at 1180. At 1160, a check is done of whether behavioral support should be provided to the user at 1160. If so, the health hub 160 may initiate communication with the user via the smartphone 150, for example. Additionally or alternately, the health hub 160 may alert the healthcare provider, via the healthcare provider interface 140, to contact the user. If not, no action is taken at 1180.

As FIG. 11 indicates, if the healthcare provider is not updated as part of any of the other actions (i.e., any of the actions at 1130-1170), an additional action that may be taken by the health hub 160, at 1190, is communication with the healthcare provider of the user via the healthcare provider interface 140. The communication may convey usage information indicating that the user is only adhering to the smoking cessation program for short periods (thus 1130 may not be reached), that the user has higher levels of nicotine or other substances than would be expected at the current stage of the smoking cessation program (thus 1190 is reach via 1160), that the user has discontinued use of the nicotine delivery device 110, or the like.

Although explanatory embodiments have been described, other embodiments are possible. Therefore, the spirit and scope of the claims should not be limited to the description of the exemplary embodiments. Various modifications and variations can be made without departing from the scope and principle of the present disclosure.

1. A smoking cessation system, comprising:
a nicotine delivery device configured to deliver a mixture in an aerosol form to a user, the mixture including nicotine, varenicline, or bupropion;
a health hub configured to communicate with the nicotine delivery device and to control operation of the nicotine delivery device based on information; and
one or more additional devices configured to communicate one or more items of the information to the health hub.

2. The smoking cessation system according to claim 1, wherein the one or more additional devices includes a smartphone used by the user.

3-4. (canceled)

5. The smoking cessation system according to claim 2, wherein the one or more items includes biometric data from the user, wherein the health hub controls the nicotine delivery device to suspend operation based on determining that the user is not an authorized user according to the biometric data.

6. The smoking cessation system according to claim 5, wherein the biometric data is a thumbprint or facial recognition.

7. The smoking cessation system according to claim 2, wherein the health hub is an application on the smartphone.

8-10. (canceled)

11. The smoking cessation system according to claim 1, wherein the one or more additional devices includes a testing device configured to obtain testing information from the user as the one or more items of the information.

12. The smoking cessation system according to claim 11, wherein the health hub controls the nicotine delivery device based on the testing information.

13. The smoking cessation system according to claim 11, wherein the testing device obtains the testing information from plasma, blood, urine, saliva, interstitial fluid, skin, sweat, breath, hair, nails, tears, ear canal, nasal passage, oral passage, anal secretion or excrement, or stool of the user, and the testing information indicates a level of nicotine, cotinine, tans-3-hydroxycotinine, nornicotine, or carbon monoxide.

14. The smoking cessation system according to claim 1, further comprising a pod configured to fit within the nicotine delivery device, wherein the mixture is stored in the pod.

15. The smoking cessation system according to claim 14, wherein the pod includes an identifier indicating an identity of the pod, the identity indicating a dosage of the mixture in the pod.

16-18. (canceled)

19. The smoking cessation system according to claim 1, wherein the nicotine delivery device provides additional information to the health hub.

20. The smoking cessation system according to claim 19, wherein the nicotine delivery device monitors a number of actuations implemented by the user and provides the number of actuations as the additional information.

21. (canceled)

22. A nicotine delivery device, comprising:
an aerosolization system configured to aerosolize a mixture for consumption by a user;
a communication interface configured to communicate with a health hub that receives information about the user; and
a processor configured to inhibit the consumption by the user based on a control signal from the health hub.

23. The nicotine delivery device according to claim 22, wherein the aerosolization system includes a mesh nebulizer, pressurized metered-dose inhaler, or soft-mist inhaler.

24. The nicotine delivery device according to claim 22, further comprising one or more measurement components, wherein one of the one or more measurement components includes a temperature sensor.

25. (canceled)

26. The nicotine delivery device according to claim 25, wherein the aerosolization system includes a temperature control system, and the temperature sensor is configured to trigger the temperature control system based on detecting an overheat condition.

27. The nicotine delivery device according to claim 24, wherein one or more of the one or more measurement components monitors an amount of the mixture consumed by the user and a number of actuations of the nicotine delivery device corresponding to dosage of aerosolized mixture taken by the user.

28. The nicotine delivery device according to claim 22, further comprising a pod interface and a pod identity reader, wherein the nicotine delivery device interfaces with a pod storing the mixture at the pod interface and the pod identity reader is configured to determine an identity of the pod.

29. The nicotine delivery device according to claim 28, wherein the identity of the pod is associated with a dosage of the mixture stored by the pod and the communication interface is used to communicate the identity of the pod to the health hub.

30. (canceled)

31. The nicotine delivery device according to claim 22, further comprising a biometric reader configured to obtain biometric information from the user.

32-49. (canceled)
